(19) United States
(12) Patent Application Publication
(10) Pub. No.: US 2025/0186306 A1
(43) Pub. Date: Jun. 12, 2025

(54) System and Method of Controlling Access to Medication with an Interactive Vial

(71) Applicant: Laila Velani, Sugar Land, TX (US)

(72) Inventor: Laila Velani, Sugar Land, TX (US)

(21) Appl. No.: 19/057,356

(22) Filed: Feb. 19, 2025

Related U.S. Application Data
(63) Continuation-in-part of application No. 16/387,375, filed on Apr. 17, 2019, now Pat. No. 12,257,212.
Application No. 16/387,375 is a continuation of application No. 15/642,433, filed on Jul. 6, 2017, now abandoned.

(51) Int. Cl. A61J 7/00; A61J 1/03; A61J 7/04; G06V 40/16; G16H 10/60; G16H 20/13; G16H 40/63

(52) U.S. Cl. CPC A61J 7/0076; A61J 1/03; A61J 7/0418; A61J 7/0472; A61J 7/0481; G16H 20/13; G16H 40/63; A61J 7/0445; A61J 2200/30; A61J 2200/72; A61J 2205/50; G06V 40/161; G06V 40/172; G16H 10/60

(57) ABSTRACT

Systems and methods for controlling access to a medication include an interactive vial communicatively connected to an offsite server storing an artificial intelligence model. The interactive vial includes a housing configured to receive and store the medication prescribed for a patient and a controllable lock configured to inhibit access into the housing for a period of time. The offsite server includes a processor configured to execute the at least one artificial intelligence model to generate a patient report, wherein executing the at least one artificial intelligence model comprises: parsing through the patient database to locate data associated with the patient; and inputting the data associated with the patient into a patient report workflow to generate the patient report.

### CROSS REFERENCE TO RELATED PATENT APPLICATIONS

The present application is a continuation-in-part claiming the benefit of U.S. patent application Ser. No. 16/387,375, filed Apr. 17, 2019, which is a continuation claiming the benefit of U.S. patent application Ser. No. 15/642,433, filed on Jul. 6, 2017, each of which are herein incorporated by reference.

### TECHNICAL FIELD

The present invention relates to an intelligent, technology-integrated pharmaceutical vial designed to enhance medication adherence, prevent misuse, and provide real-time insights into patient medication habits. This invention is applicable to all patient populations, including those with dementia, disabilities, opioid prescriptions, and chronic conditions requiring strict adherence. Additionally, the present invention is designed to be recyclable and reusable, reducing pharmaceutical waste while maintaining secure data tracking.

### BACKGROUND

For all medications, controlled and/or non-controlled, the current opioid epidemic starts at home by abusing prescribed medications. Typically, a patient abuses medication by taking too many pills too close together ahead of the prescribed medication dosage schedule. In other cases, children access prescription medications and become addicted opioids and turn to street drugs to but illicit drugs. Thus, there is a need for a medication dispensation system and method that controls access by medications at unauthorized times ahead of a medication dosage schedule or by persons other than the patient.

Medication non-adherence is a critical issue, leading to worsening health conditions, hospitalizations, and even fatalities. Many patients, particularly those with dementia or cognitive impairments, struggle to take their medication on time. Additionally, the opioid crisis has highlighted the urgent need for controlled dispensing to prevent overdoses and misuse. Current medication vials offer no interactive or preventive measures, relying solely on patients to self-regulate their intake.

There is also an environmental concern regarding single-use plastic pharmaceutical vials, which contribute significantly to medical waste. There is a need for a vial being fully recyclable and reusable within a closed-loop system involving pharmacies and healthcare providers.

Advances in artificial intelligence models have allowed users to better analyze data for desired results. These models can be utilized by medical providers to better predict when a patient may be abusing their prescription medicine, as well as predict the effectiveness of the medication in treating the patient's illness.

### BRIEF DESCRIPTION OF THE DRAWINGS

For a more complete understanding of the present disclosure and its features and advantages, reference is now made to the following description, taken in conjunction with the accompanying drawings, in which:

FIG. 1 is block diagram of interactive vial according to one embodiment of the invention;

FIG. 2 is diagram of a patient data stored in data structure for used by a processor accessing the data structure in a computer readable medium for controlling dispensing of a prescribed medication;

FIG. 3 is diagram of a patient data stored in data structure for used by a processor accessing the data structure in a computer readable medium for controlling dispensing of a prescribed medication;

FIGS. 4A-4C are flow charts for controlling dispensing of medication;

FIG. 5 is general diagram of a computer system for use in a particular embodiment of the invention; and

FIG. 6 is a block diagram of the medication dispensing system according to one embodiment of the invention.

### DETAILED DESCRIPTION

Illustrative embodiments of the present disclosure are described in detail herein. In the interest of clarity, not all features of an actual implementation are described in this specification. It will of course be appreciated that in the development of any such actual embodiment, numerous implementation specific decisions must be made to achieve developers' specific goals, such as compliance with system related and business related constraints, which will vary from one implementation to another. Moreover, it will be appreciated that such a development effort might be complex and time consuming, but would nevertheless be a routine undertaking for those of ordinary skill in the art having the benefit of the present disclosure. Furthermore, in no way should the following examples be read to limit, or define, the scope of the disclosure.

A Smart Vial interactive medication dosage dispensing system and method, also referred to herein as a “Smart Vial” or “smart vial” is disclosed that dispenses controlled and/or non-controlled medications as prescribed by a licensed medical professional such a physician. In a particular embodiment, the Smart Vial includes but is not limited to a Smart electronic vial for containing and delivering controlled and/or non-control medications as directed by the doctor to a particular patient. An alarm on the digital vial is used to announce delivery of controlled substances and violation of a dosage schedule. A thumb print and/or access code is to release the non-control substances which also uses voice print, iris pattern, gesture, sweat print, double authentication send password to vial, have user speak password and compare to voice print for user. The Smart Vial reduces misuse of medication by kids. The Smart Vial provides a USB digital data port and wireless input output that enables a doctor, pharmacy, emergency medical personnel or patient to access the patient data stored in a data base. A doctor can check the patient data which includes but is not limited to a dispensary report (indicating what medications a patient has taken, dosage and time taken), patient prescription history and/or medication history. In a particular embodiment software is provided comprising computer executable instruction stored on a computer readable medium in data communication with the Smart Vial processor that enables a pharmacy and doctor to fill a medical prescription and to run a dispensary report and to check whether the patient is taking medication on schedule or not. “Data communication” is used herein to mean data is exchanged in both directions between two devices that are in “data communication”.

The Smart Vial is a revolutionary step in medication management, offering enhanced security, improved adherence, overdose prevention, and real-time healthcare insights. Its recyclability may ensure sustainability, making it a cost-effective, environmentally-friendly, and patient-centric solution. The Smart Vial may improve medication adherence and safety for a broad range of individuals, including: dementia patients who struggle with memory loss and need structured reminders; individuals with disabilities who require assistance with medication management; long-term mental health patients, such as those with bipolar disorder or schizophrenia, where adherence is crucial to stability; and/or chronic condition patients (e.g., heart disease, diabetes, hypertension) where medication compliance directly impacts treatment effectiveness.

In certain embodiments, the Smart Vial may be a secure, AI-powered, recyclable medication vial with multiple functionalities designed to enhance patient safety, adherence, and healthcare insights. Without limitations, certain features include: 1). timed dispensing & access control: wherein medication may be accessible only at prescribed times via biometric authentication (i.e., eye retina scanner and thumbprint scanning), and wherein the Smart Vial may prevent early access or unauthorized use which is crucial for opioid prescriptions and controlled substances; 2). medication adherence monitoring: wherein integrated AI may track vial openings and alert healthcare providers if the medication is not taken as prescribed, and wherein the Smart Vial may help doctors and insurance companies assess whether treatment is effective or needs adjustment; 3). overdose & misuse prevention: wherein if a patient attempts excessive access, the SmartVial may lock itself to prevent potential overdose, and wherein alerts can be sent to caregivers, physicians, or emergency contacts in cases of suspected misuse; 4). recyclability & reusability: wherein the Smart Vial can be returned to pharmacies or doctor's offices each month for data reset and sterilization, reducing plastic waste, and wherein the Smart Vial may maintain secure patient-specific data encryption to protect privacy while allowing authorized reuse; 5). multi-patient applications (beyond opioid control): for dementia patients & disability support: automatic audible and visual alerts remind patients to take their medication; for nursing homes & home care: caregivers can track adherence without manual logging; and for heart patients & chronic disease management: real-time data allows doctors to assess whether lack of improvement is due to non-adherence or ineffective treatment; 6). smart reminders & emergency alerts: wherein if a patient forgets a dose, the vial may trigger alarms, phone notifications, or caregiver alerts, and wherein if doses are consistently missed, physicians are automatically notified to reassess treatment; and 7). integration with healthcare & insurance systems: wherein data analytics enable insurance providers and doctors to confirm whether a patient is following the prescribed regimen, and wherein the Smart Vial may help optimize medication plans, reduce unnecessary prescriptions, and prevent insurance fraud.

In a particular embodiment, a Wi-Fi device or smart phone is connected to the Smart Vial through which the doctor can get a report of the patient data. The Smart Vial data base contains patient data including but not limited to Patient DL and ID allergy/DOB/Doctor/patient name, age, weight, medical history, food consumption, medication dosage history indication what medications dosages a patient has taken and when (time of day) and pharmacy records. Medications that are stored in the Smart Vial can only be accessed by the patient during the medication time (not before directed medication time). An alert is selectively sent to the provider if the patient is taking overdose, accidentally taking the prescribed medications. The Smart Vial is a reusable and refillable vial. In a particular embodiment, the Smart Vial processor monitors the patient data in the data to calculate a sliding scale drug interaction warning and dosage adjustment based on timing and dosage based on biometric data of patient and timing of dosage. In a particular embodiment, the Smart Vial audibly announces a food warning prior to dosage time, based on a sliding scale calculated by accessing the patient data to indicate what the patient has eaten and what medication the patient has taken. For example, if a patient data indicates the patient has not eaten within 30 minutes of when the dosage is due, the Smart Vial announces that the patient needs to cat and enter the data in the patient data before the dosage is released with a YES command to unlock the vial. If the patient data indicates the patient has eaten within 30 minutes of when the dosage is due, the Smart Vial the dosage is released with a YES command to unlock the vial. If a patient data indicates the patient has not eaten within 30 minutes of when the dosage is due, the Smart Vial announces that the patient needs to eat and enter the data in the patient data before the dosage is released with a YES command to unlock the vial. If the patient data indicates that the dosage requires no food more than 30 minutes before taking, and the medication requires no food to be eaten with the medication, the smart vial announces that the patient should wait to take the medication (also referred to herein as “dosage”) for a period of time indicated in the patient data for the medication as indicated in the physician's and pharmacy's data base and sent to the patient data.

In another particular embodiment, the Smart Vial contains a digital camera to take an image of a patient's face to confirm the identity of a patient and also to take an image of a medication being released by the smart vial to confirm the identity of a pill the patient is taking. The pill identity of the pill is confirmed with the patient data base as the pills are placed into the Smart Vial and again as the pill exits the smart vial. The pill is not released by the Smart Vial if the identity of the pill is not confirmed with the patient data on exit from the smart vial. The Smart Vial processor identifies the medication/pills image by comparing the medication image taken by the camera at the Smart Vial to a reference image in the patient data provided by the pharmacy or doctor or medication manufacturer for the prescribed medicine using the shape, color, logo and number on Smart Vial as compared to the reference image in the patient data for the medications prescribed to the patient and contained in the smart vial. If the any one of the shape, color, logo or number on the Smart Vial image of the pill does not match the reference image for prescribed pill, dispensing of the pill is blocked by the Smart Vial processor. The pharmacy and doctor are contacted and are sent the image of the pill being dispensed and the reference image to confirm or override the block on the dispensing of the pill. Errors can occur before the medication is sent to patient in the Smart Vial. An error can occur if the doctor leaves off a critical part of the prescription such as a suffix on a particular medication when calling in a prescription to a pharmacy or the pharmacist may not record the prescription properly leaving off a critical part of the prescription such as a suffix on a particular medication, or a number. Such errors by the doctor or pharmacy can cause the wrong medicine to be loaded into the Smart Vial. Thus, the actual dispensed medicine is imaged by the Smart Vial and checked before dispensing a medication. The pill is identified and compared to the reference image on the Smart Vial to confirm that the type of medication and dosage are verified as proper before dispensing the medication.

In a particular illustrative embodiment, the Smart Vial processor manages and dispenses multiple pills having multiple pill delivery schedules in conjunction with a sliding scale drug interaction and food regiment.

The Smart Vial is made of natural or un-natural material (e.g., plastic, metal). The Smart Vial helps to reduce addiction and overdose and the epidemic on controlled substance abuse. The Smart Vial is useful for doctors, pharmacy, probation officers, court systems to verify whether the patient is taking their prescribed medications on schedule. In certain embodiments, the Smart Vial may be returned to pharmacies or a doctor's office each month for data reset and sterilization, reducing plastic waste and promoting re-use and recyclability.

In another embodiment, the Smart Vial camera is used to capture video of the patient to monitor and confirm that patient places the imaged pill on their tongue and shows that the pill has been swallowed by the patient by imaging sending a video stream to a Smart Vial server and showing the imaged and dispensed pill on the patient's tongue is no longer visible after being placed on the tongue and drinking water. The Smart Vial helps to continuously track the usage of drugs by the patient.

In a particular illustrative embodiment, the Smart Vial system and method accepts patient authentication data to verify a user's identity. The authentication data includes but is not limited to a patient's iris image captured by the camera for comparison to an iris reference image for the patient, a patient's finger print image captured by the camera for comparison fingerprint reference image, a patient's sweat print captured by the camera for comparison sweat print reference signature, a patient's voice recording captured by the camera for comparison voice print reference which are entered concurrently.

In another embodiment, the Smart Vial generates a password that is spoken by the patient to access the data. The Smart Vial processor accesses the patient data including in the data base, the patient data including but not limited to medical history, age, weight, biometrics for blood pressure, respirations, pulse, blood sugar level, food eaten and when, pills taken, dosage and when and pending dosage schedule. The system and method determines a sliding scale dosage time and quantity and food requirement based on the patient data. The patient data is contained in a medical data base for all patients which can be accessed by police, physicians and emergency personnel when a patient is unconscious or otherwise incapacitated.

The patient can enter additional patient data including but not limited to a pain level to indicate a level of urgency to be taken into account for timing and dosage, for example, in an urgent situation where the pain level is 8 or 9 on a scale of ten, the dosage can be increased with additional pills and the dosage time can be advanced so that the pill is available sooner than the normal dosage schedule if not immediately. or anxiety level to indicate high anxiety. The patient, can enter acute symptoms or and biometric data input into the system processor to determine a sliding scale adjustment to the dosage schedule based on the patient data. The patient also enters foods eaten, quantity of food eaten and the times the foods were eaten as patient data to entering into determining the sliding scale adjustment to the dosage schedule based on the patient data. The system uses the patient data to announce food requirements and prohibitions for the patient.

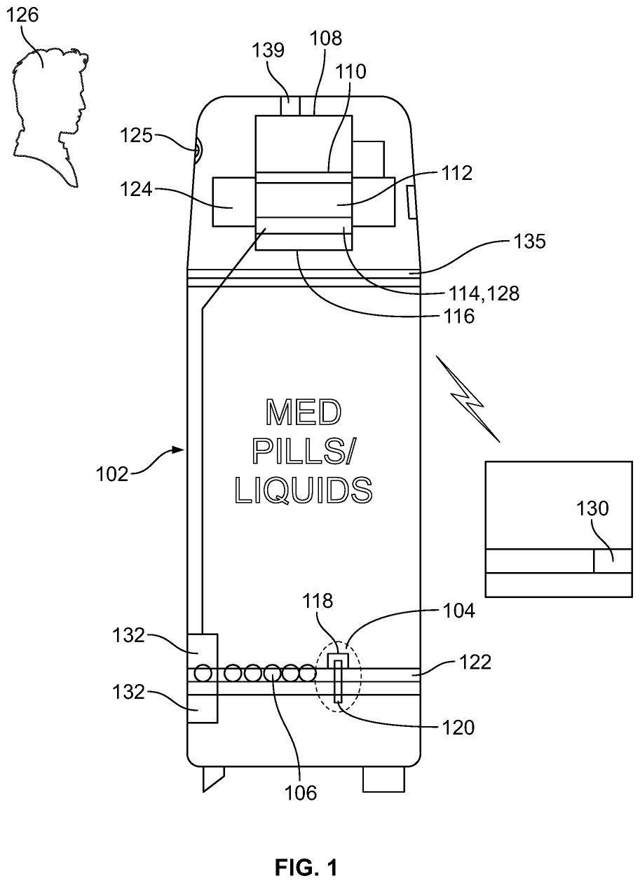

Turning now to FIG. 1, in a particular illustrative embodiment a medication dispensing system 100 includes but is not limited to an interactive vial 102 having a controllable lock 104 for containing medications 106 prescribed for a patient. In certain embodiments, the interactive vial 102 may comprise a housing constructed from transparent material. A processor 108 is contained in the vial. A computer program 110 stored in a computer readable medium 112 the computer program comprising instructions for execution by the processor, the computer program including but not limited to instructions to read patient data 114 for the patient from a data base 116 stored in the computer readable medium 112, instructions to authorize dispensing of the medication from the vial; and instructions to upon reading an authorization value of “YES”, unlock the vial to allow access to the medication from the vial. In a particular illustrative embodiment the controllable lock is a magnetically actuated metal pin 116 and solenoid 118 to magnetically lift the metal pin from a rotatable pill container 120, the instructions to unlock the vial further comprising instructions to send an unlock command to the controllable lock to lift the magnetically actuated pin 116 out of a receiving hole 120 on the rotatable pill container 122 to allow the rotatable pill container to rotate and provide access to a single dosage of the medication from the vial. The computer program further comprises instructions to send a lock command to the lock to release and lower the magnetically actuated pin into the receiving hole 120 on the rotatable pill container 122 to resist further rotation of the rotatable pill container so that only a single dosage of the medication 123 is dispensed.

In a particular embodiment, the system of claim 1, the vial further comprises a patient authentication camera 124 that takes a picture of the patient's face 126; a patient's face reference image 128 stored in patient data 130 for the patient in a database 116. In a particular embodiment, the computer program further comprising instructions to capture and store in the data base an image of the patient's face (picture) 126 on the vial patient authentication camera; instructions to compare the compare the image of the patient's face from the vial camera to the face reference image 128. In another embodiment, the computer program further comprises, instructions to, when the an image of the patient's face on the vial camera and the reference image are compared and correlate above 90, instructions to generate store in the data base a facial recognition authentication result with a value 130 of “YES” indicating a positive result indicating that the vial camera image and the reference image match, else instructions to generate and store in the data base a facial recognition authentication result with a value of “NO” indicating negative result indicating a positive result indicating that the vial camera image and the reference image do not match.

In another particular embodiment, the system further comprises a pair of medication authentication cameras 132 that takes top and bottom pictures (images) 131 of the single dosage of medication 123 in the vial before setting a medical verification value 130 to “YES” indicating that the medication matches a doctor's reference image 140 of the prescribed medication stored in the data base. The doctors reference image is coded at the doctor's office computer which then sends electronically the doctors reference images placed in the patient's data in the data. In another embodiment, the computer program further comprises instructions to compare the compare one or both of the images of the single dose of medication 123 taken by the vial camera, to the medication reference image 140 in the data base and instructions to when the an image of the single dose of medication on the medication authentication camera 132 and the reference image correlate above 90 percent matching, generate and store in the data base a medication authentication result with a value of “YES” indicating a positive result indicating that the vial camera images and the reference image match, else generate and store in the data base a facial recognition authentication result with a value of “NO” indicating negative result indicating a positive result indicating that the vial camera image and the reference image do not match.

In another embodiment, system of further comprises a patient authentication camera that takes a video of the patient's face taking the medication showing the medication on the patient's tongue and then swallowed by the patient and streams the video to the patient data in the data base, in a particular embodiment the database is on a server and the video is streamed to the server.

The data also contains a reference video of the patient's face taking the medication stored in patient data for the patient in a database, the computer program further comprising instructions to compare the video 134 of the patient's face taking the medication to the reference video 136 when the video of image of the patient's face on the vial camera and the reference of the patient's face taking the medication correlate above 90 percent matching, generate store in the data base a medication taken authentication result with a value of “YES” indicating a positive result indicating that the of the patient's face taking the medication and the reference video match and the patient has taken the medication, else generate store in the data base a medication taken authentication result with a value of “NO” indicating a positive result indicating that the of the patient's face taking the medication and the reference video match and the patient has not taken the medication.

In another embodiment, the system further comprises a temperature sensor on the vial, the computer program further comprising instructions to monitor the temperature sensor to determine a vial temperature and store a vial temperature history in the patient data showing vial temperature measurements and the time at which the temperature measurements were made, in one example the temperature is measured once an hour; and instructions to issue an alarm when the vial temperature exceeds 80 degrees Fahrenheit or the vial temperature history exceed a manufacturer's recommendation for storage temperature for the prescribed medication. In some embodiments, the system may further comprise at least one quantum temperature sensor configured to measure the temperature of the interactive vial 102.

In another embodiment, the system further comprises instructions to read patient data from the data base for medications the patient has taken and when; and instructions to calculate, using drug interaction stored in the data base for the medication for the patient, a sliding scale medication dosage schedule based on the food eaten and when and the medications taken and store the sliding scale food requirement value in the data base as an authentication value parameter for calculating the authentication value to unlock the vial.

In another embodiment, the system further comprises instructions to read patient data for food the patient has eaten stored in the patient data as input by the patient or nurse; and instructions to calculate, using doctor food recommendations stored in the data base for the medication for the patient, a sliding scale food requirement value based on food eaten by the patient and when and the medications taken and store the sliding scale food requirement value in the data base as an authentication value parameter for calculating the authentication value to unlock the vial.

In another embodiment, the database is stored in a computer readable medium on the server; the computer program further comprising instructions to determine when a seal 135 on the vial has been breached, instructions to send an alert to authorities when the vial has been breached. A speaker 139 on the vial emits an alarm signal when the vial is open outside of the pharmacy. In another embodiment, the system of the computer program further comprises instructions to sending the unlock command, followed by user setting of delay of 2 to 10 seconds later, at the end of the delay a lock command to resist rotation of the medication wheel. The vial processor send the unlock command to the solenoid that raises the metal pin stopping rotation of the medication wheel.

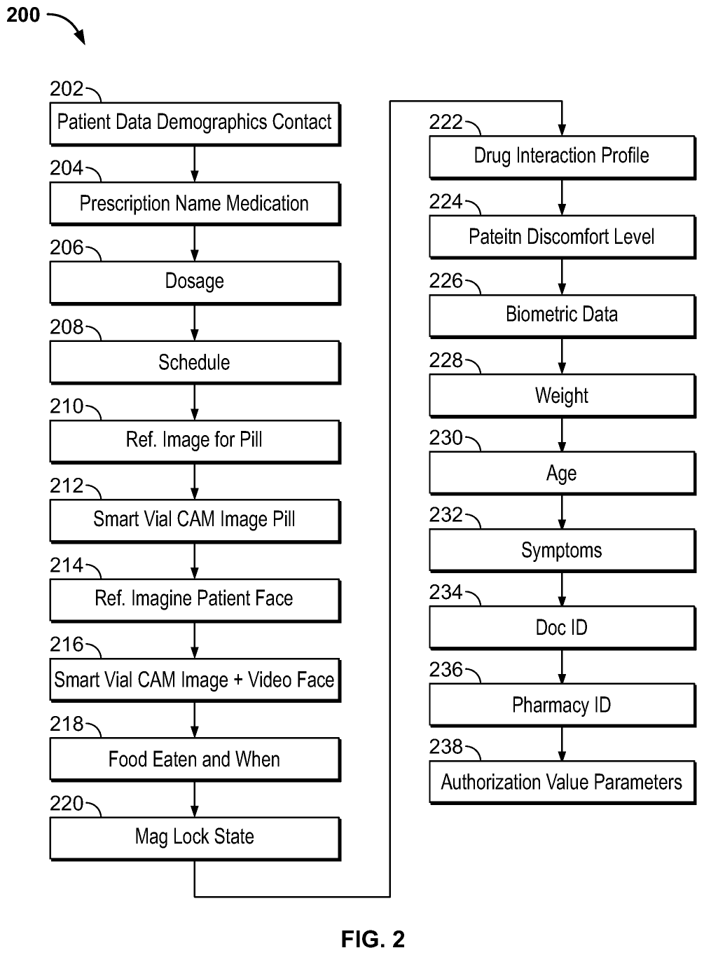

Turning now to FIG. 2, in a particular illustrative embodiment, a data structure 200 containing patient data 202 used by the vial processor to control dispensing of the medication. As shown in FIG. 2, the data structure includes fields containing patient data accessed and used by the vial processor to control dispensing of the medication. Field 202 contains patient data, prescription name 204, medication dosage 206 and dosage schedule 208, 210 reference images of the top and bottom images of the medication, 212 medication camera top and bottom images, 214 reference images and reference video of patient taking medication, 216 face authentication camera facial image and video of patient taking medication; 218 patient food eaten and when eaten; 220 magnetically actuated lock state: “locked/unlocked”. 222 drug interaction profile based on medications taken, when taken and food eaten and when; 224 patient discomfort level; 226 patient biometric data; 228 patient weight; 230 patient age; 232 patient symptoms; 234 prescribing doctor's identification; 236 medication originating pharmacy identification and 238 authorization value parameters. The drug interaction profile for the patient is stored in the data base.

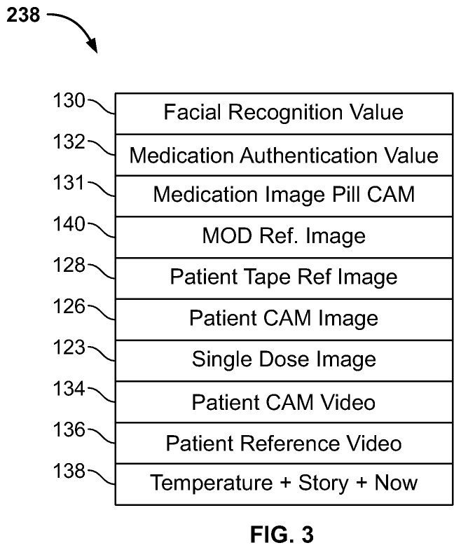

As shown in FIG. 3 field 238 further comprises facial recognition value 130, medication authorization value 132, 131 medication images of the dosage pill taken from the medication cameras on the vial, 140 medication reference images from doctor; patient face reference image 128; patient authentication camera image 126; single dose images (top and bottom) 123; patient authentication camera video 134; patient reference video 136 and vial temperature and vial temperature history.

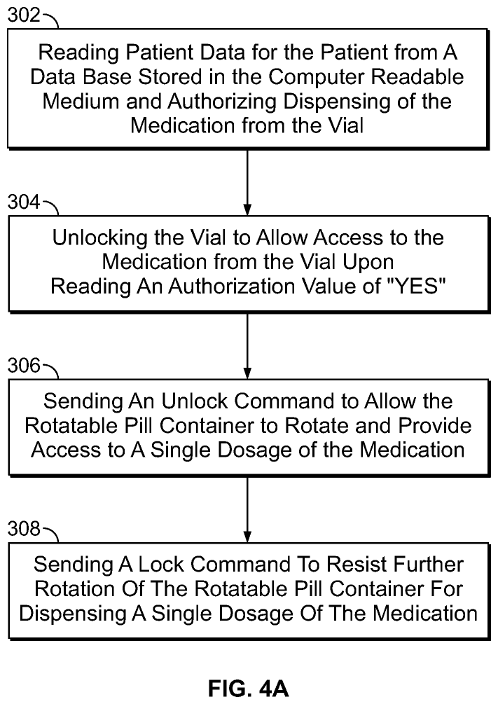

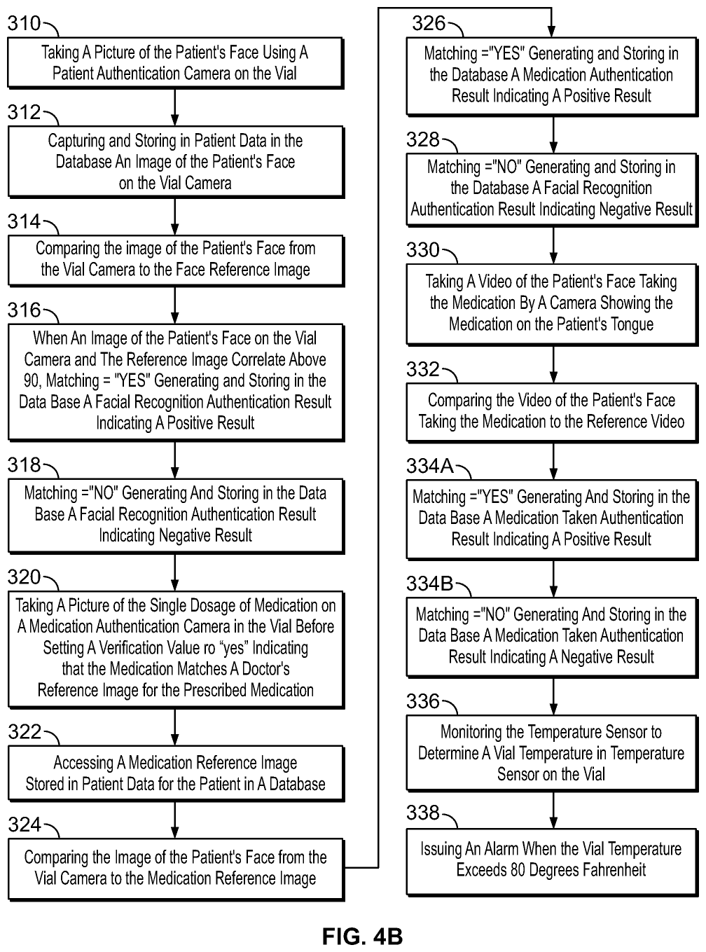

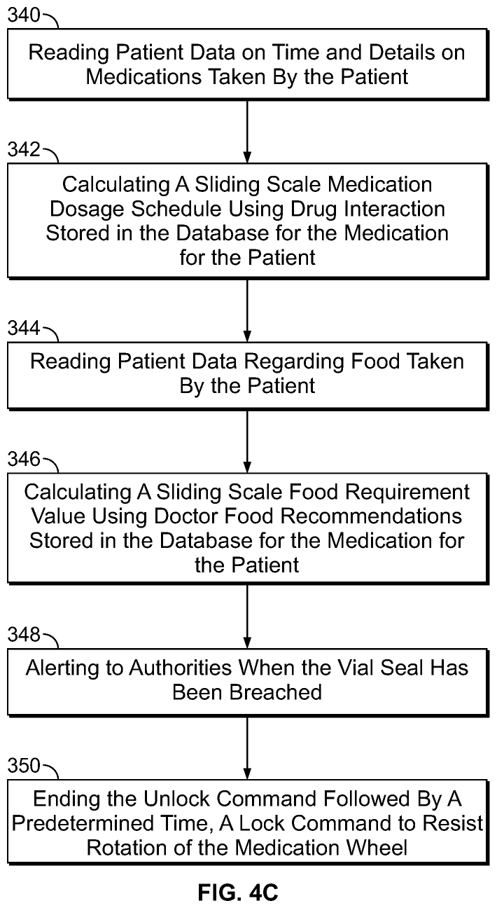

With reference to FIGS. 4A-4C, the method starts at 302 reading patient data for the patient from a data base stored in the computer readable medium; 302 authorizing dispensing of the medication from the vial; and 304 upon reading an authorization value of “YES”, unlocking the vial to allow access to the medication from the vial. The method proceeds to 306 sending an unlock command to the controllable lock to lift a magnetically actuated pin out of a receiving hole on the rotatable pill container to allow the rotatable pill container to rotate and provide access to a single dosage of the medication and 308 sending a lock command to the lock to release and lower the magnetically actuated pin into a receiving hole on the rotatable pill container to resist further rotation of the rotatable pill container so that only a single dosage of the medication is dispensed.

In another embodiment the method further comprises 310 taking a picture of the patient's face using a patient authentication camera on the vial; 312 capturing and storing in patient data in the data base an image of the patient's face on the vial camera; and 314 comparing the compare the image of the patient's face from the vial camera to the face reference image; and 316 when the an image of the patient's face on the vial camera and the reference image correlate above 90 percent matching, generating and storing in the data base a facial recognition authentication result with a value of “YES” indicating a positive result indicating that the vial camera image and the reference image match, else 318 generating and storing in the data base a facial recognition authentication result with a value of “NO” indicating negative result indicating a positive result indicating that the vial camera image and the reference image do not match.

In another embodiment the method further comprises 320 taking a picture of the single dosage of medication on a medication authentication camera in the vial before setting a verification value to “YES” indicating that the medication matches a doctor's reference image for the prescribed medication; 322 accessing a medication reference image stored in patient data for the patient in a database; 324 comparing the image of the patient's face from the vial camera to the medication reference image; 326 generating and storing in the data base a medication authentication result with a value of “YES” indicating a positive result indicating that the medication authentication camera image and the medication reference image match when the an image of the patient's face on the vial camera and the reference image correlate above 90; else 328 generating and storing in the data base a facial recognition authentication result with a value of “NO” indicating negative result indicating a positive result indicating that the vial camera image and the reference image do not match. In another embodiment the “YES” and “NO” commands are markers inserted into an authentication data stream sent to the vial processor. A YES marker comprises two YES markers comprising a data value of 1 are sent 100 milliseconds apart from each other which are acknowledged by the vial processor, followed by two second additional YES markers comprising a data value of 2 are sent to the vial processor separated by 200 milliseconds after vial processor acknowledges the first two YES markers. If the timing or data value varies for either the first two YES markers or the second two YES markers, the YES command sequence is ignored by the vial processor.

A NO marker comprises two NO markers comprising a data value of 3 are sent 50 milliseconds apart from each other which are acknowledged by the vial processor, followed by two second additional NO markers comprising a data value of 4 are sent to the vial processor separated by 250 milliseconds after vial processor acknowledges the first two NO markers. If the timing or data value varies for either the first two NO markers or the second two NO markers, the NO command sequence is ignored by the vial processor.

In another embodiment the method further comprises 330 taking a video on the patient authentication camera, of the patient's face taking the medication showing the medication on the patient's tongue and then swallowed; 332 comparing the video of the patient's face taking the medication to the reference video when the video of image of the patient's face on the vial camera and the reference video of the patient's face taking the medication correlate above 90, 334 generating and storing in the data base a medication taken authentication result with a value of “YES” indicating a positive result indicating that the of the patient's face taking the medication and the reference video match and the patient has taken the medication, else generating and storing in the data base a medication taken authentication result with a value of “NO” indicating a positive result indicating that the of the patient's face taking the medication and the reference video match and the patient has not taken the medication. The method further proceeds to 336 monitoring the temperature sensor to determine a vial temperature a temperature sensor on the vial; and 338 issuing an alarm when the vial temperature exceeds an alarm temperature of 80 degrees Fahrenheit. The alarm temperature is programmable and is stored in the physician's data base and pharmacy's data based and is down loaded to memory accessed by the vial processor. The vial processor issues and alarm when the vial temperature exceeds the alarm temperature for a duration specified in the database and down loaded to memory accessed by the vial processor. In some embodiments, the interactive vial 102 may further comprise an internal alarm notification system to alter the patient of a missed dosage.

In another embodiment, the method further comprises 340 reading patient data for medications the patient has taken and when the medications were taken; and 342 calculating, using drug interaction stored in the data base for the medication for the patient, a sliding scale medication dosage schedule based on the food eaten and when and the medications were taken and store the sliding scale food requirement value in the data base as an authentication value parameter for calculating the authentication value to unlock the vial.

In another embodiment, the method further comprises 344 reading patient data for food the patient has eaten; and 346 calculating, using doctor food recommendations stored in the data base for the medication for the patient, a sliding scale food requirement value based on food eaten and when and the medications taken and store the sliding scale food requirement value in the data base as an authentication value parameter for calculating the authentication value to unlock the vial. In another embodiment, the vial processor uses the sliding scale food requirement to determine and announce at the vial as an alert to the patient taking the medication that they need to cat while taking the medication.

In another embodiment, the method further comprises 348 alerting to authorities when the vial seal has been breached. In another embodiment, the method further comprises 350 ending the unlock command followed by 2 seconds later, a lock command to resist rotation of the medication wheel.

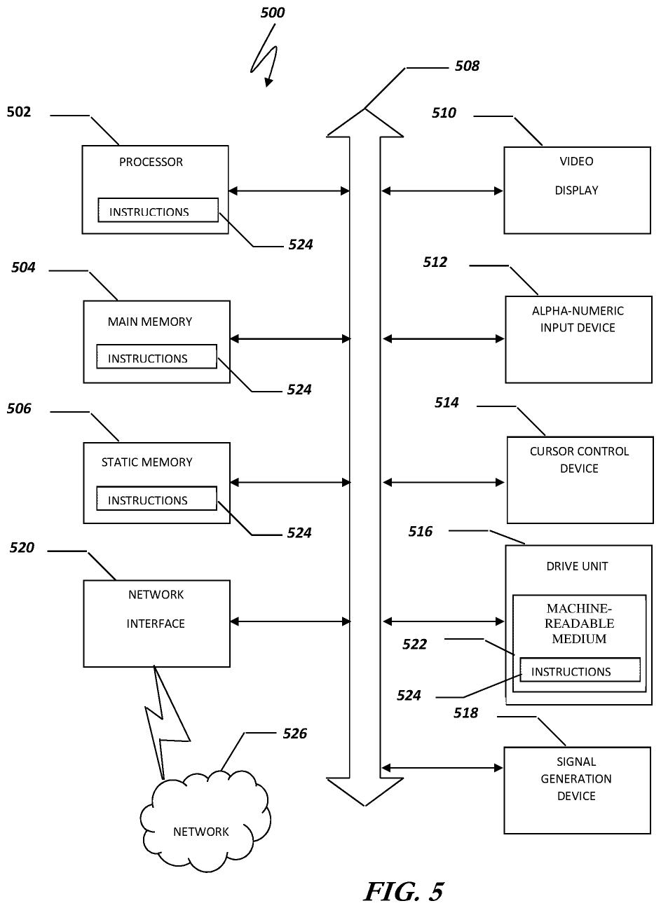

FIG. 5 is a diagrammatic representation of a machine in the form of a computer system 500 within which a set of instructions, when executed, may cause the machine to perform any one or more of the methodologies discussed herein. In some embodiments, the machine operates as a standalone device. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment. The machine may comprise a server computer, a client user computer, a personal computer (PC), a tablet PC, a set-top box (STB), a Personal Digital Assistant (PDA), a cellular telephone, a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any machine capable of executing a set of instructions (sequential or otherwise) that specify actions to be taken by that machine.

It will be understood that a device of the present invention includes broadly any electronic device that provides voice, video or data communication. Further, while a single machine is illustrated, the term “machine” shall also be taken to include any collection of machines that individually or jointly execute a set (or multiple sets) of instructions to perform any one or more of the methodologies discussed herein.

The Smart Vial includes but is not limited to a computer system 500 may include a Smart Vial processor 502 (e.g., a central processing unit (CPU), a graphics processing unit (GPU), or both), a main memory 504 and a static memory 506, which communicate with each other via a bus 508. The computer system 500 may further include a video display unit 510 (e.g., liquid crystals display (LCD), a flat panel, a solid-state display, or a cathode ray tube (CRT)). In embodiments, processor 502 may be operable to transmit the data associated with the patient. In some embodiments, the processor 502 may utilize quantum encryption to protect patient data. In certain embodiments, the display unit 510 may be configured to display one or more alerts, including but not limited to, a dosage schedule, a temperature of the interactive vial, and/or any combination thereof. The computer system 500 may include an input device 512 (e.g., a keyboard), a cursor control device 514 (e.g., a mouse), a disk drive unit 516, a signal generation device 518 (e.g., a speaker or remote control) and a network interface.

The disk drive unit 516 may include a machine-readable medium 522 on which is stored one or more sets of instructions (e.g., software 524) embodying any one or more of the methodologies or functions described herein, including those methods illustrated in herein above. The instructions 524 may also reside, completely or at least partially, within the main memory 504, the static memory 506, and/or within the processor 502 during execution thereof by the computer system 500. The main memory 504 and the processor 502 also may constitute machine-readable media. Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present invention, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

As shown in FIG. 5, the present invention contemplates a machine-readable medium containing instructions 524, or that which receives and executes instructions 524 from a propagated signal so that a device connected to a network environment 526 can send or receive voice, video or data, and to communicate over the network 526 using the instructions 524. The instructions 524 may further be transmitted or received over a network 526 via the network interface device 520. The machine-readable medium may also contain a data structure for containing data useful in providing a functional relationship between the data and a machine or computer in an illustrative embodiment of the disclosed system and method.

While the machine-readable medium 522 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. The term “machine-readable medium” shall accordingly be taken to include, but not be limited to: solid-state memories such as a memory card or other package that houses one or more read-only (non-volatile) memories, random access memories, or other re-writable (volatile) memories; magneto-optical or optical medium such as a disk or tape embodying computer instructions in a transmission medium; and/or a digital file attachment to e-mail or other self-contained information archive or set of archives is considered a distribution medium equivalent to a tangible storage medium. Accordingly, the invention is considered to include any one or more of a tangible machine-readable medium or a tangible distribution medium, as listed herein and including art-recognized equivalents and successor media, in which the software implementations herein are stored.

Although the present specification describes components and functions implemented in the embodiments with reference to particular standards and protocols, the invention is not limited to such standards and protocols. Each of the standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same functions are considered equivalents.

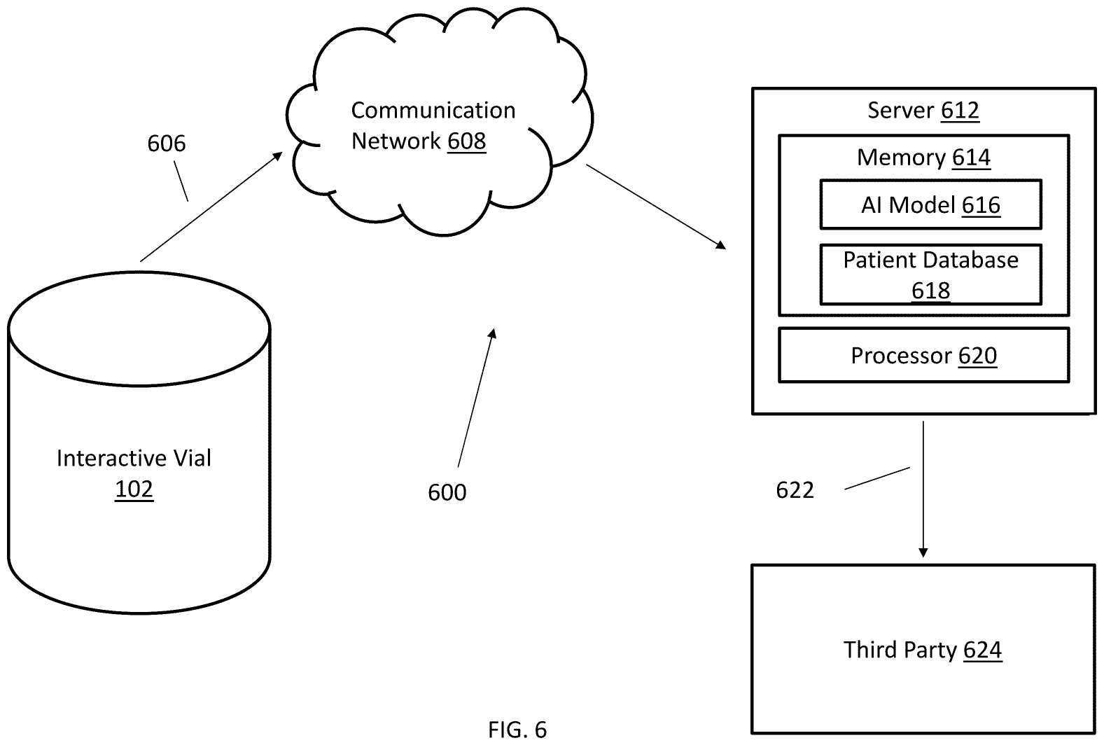

FIG. 6 illustrates an example medication dispensing system 600. In one or more embodiments, the medication dispensing system 600 may include the interactive vial 102 (with reference to FIGS. 1-5) that may be communicatively coupled to an offsite server 612 through a communication network 608. The offsite server 612 may comprise a memory 614, where the memory 614 may store at least one artificial intelligence (AI) model 616. The memory 614 may further store a patient database 618, which may comprise data 606 received from the interactive vial 102. The memory 614 may be communicatively coupled to a processor 620. The processor 620 may execute the artificial intelligence model 616 by parsing through the patient data stored in the patient database 618 and inputting the patient data into a patient report workflow to generate a patient report 622.

The processor 620 may be configured to process raw data (i.e., data 606) for input to the AI model 616. This processor 620 may be configured to clean, normalize, and/or encode the data 606 to ensure it is in a format compatible with the requirements of the AI model 616. The processor may be configured to handle operations such as filling missing values, scaling numerical features, and encoding categorical data.

The AI model 616 may comprise architecture designed to process input data and generate predictions or classifications through a workflow. In one embodiment, the architecture may include neural network layers such as convolutional layers, recurrent layers, or fully connected layers, depending on the workflow. The AI model 616 may be trained or untrained. In embodiments wherein AI model 616 is trained, a dataset that includes labeled examples may be used. The training process may include adjusting the model's internal parameters to minimize the error between the model's predictions and the actual values. The training process may utilize algorithms such as gradient descent to optimize these weights iteratively. A large dataset, often split into training, validation, and test sets, may be used to ensure that the AI model 616 can sufficiently generalize unseen data.

Once trained, the AI model 616 may be in the inference phase, wherein it processes new, unseen data to make predictions. During inference, the AI model 616 may apply the learned weights to incoming data (i.e., data 606) and generate a prediction or output. Without limitations, the generated output may be the patient report 622. The server 612 may be configured to handle varying input data and generate output that is actionable, such as a classification label or a numerical prediction.

The AI model 616 may be accessible via an API, which provides an interface for third-party systems to communicate with the AI model 616. The API may accept data requests, process the data through the AI model 616, and return the result to the requesting system. The API may support HTTP methods such as POST, GET, and PUT, where POST requests may be used to send data to the AI model 616 and receive predictions in response.

Requests to the AI model 616 may be received by the server 612, wherein server 612 may be configured to handle API calls. Upon receiving a request, the server 612 may perform input validation to ensure the integrity of the data 606 before passing it to the AI model 616 for inference. The server 612 may then return the inference results to the third-party system that initiated the request. As illustrated, server 612 may transmit a patient report 622 to a third party 624. Without limitations, the third party 624 may be a doctor, a pharmacy, an emergency room, and a federal agency.

Over time, the AI model 616 may need to be updated or retrained to account for new data or changing patterns. The system 600 is configured to allow for periodic updates to the AI model 616 without disrupting its operation. Retraining may be done with updated datasets, and AI model 616 may be deployed with minimal downtime. This may allow the system 600 to adapt to changes in the environment or patient behavior and continue to provide accurate predictions.

The invention provides a system and method for integrating an AI model with third-party servers, enabling secure, efficient, and scalable communication. The use of preprocessing, API integration, asynchronous communication, and security mechanisms ensures that the AI model can be reliably deployed in a variety of environments. The system is designed to be adaptable, ensuring that it can accommodate varying workloads, provide real-time predictions, and securely manage sensitive data.

In embodiments, the artificial intelligence model 616 may further comprise instructions to cross reference the data associated with the patient from the patient database with a global medical database to suggest improvements in a patient treatment plan, instructions to predict when the patient needs to be notified about taking the prescribed medication, and instructions to predict when the patient report needs to be communicated to the third party 624. In these embodiments, this may be implemented in a separate workflow.

Although the present disclosure and its advantages have been described in detail, it should be understood that various changes, substitutions and alterations can be made herein without departing from the spirit and scope of the disclosure as defined by the following claims.

In the foregoing specification, embodiments of the present disclosure have been described with reference to numerous specific details that may vary from implementation to implementation. The specification and drawings are, accordingly, to be regarded in an illustrative rather than a restrictive sense. The sole and exclusive indicator of the scope of the present disclosure, and what is intended by the applicants to be the scope of the present disclosure, is the literal and equivalent scope of the set of claims that issue from this application, in the specific form in which such claims issue, including any subsequent correction.

1. A system for controlling access to a medication, the system comprising:
an interactive vial comprising:
a housing configured to receive and store the medication prescribed for a patient;
a controllable lock configured to inhibit access into the housing for a period of time; and
a pair of medication authentication cameras coupled to the interactive vial that take a picture of a front side and a back side of a single dosage of medication in the interactive vial and comparing the front side and the back side pictures to reference images for a medication shape, color and logo, and number on the front side and the back side pictures before setting a verification value to “YES” indicating that the medication matches the reference image for the prescribed medication; and
an offsite server communicatively coupled to the interactive vial through a communication network, the offsite server comprising:
a memory configured to store:
at least one artificial intelligence model; and
a patient database comprising data received by the interactive vial; and
a processor, communicatively coupled to the memory, configured to:
execute the at least one artificial intelligence model to generate a patient report, wherein executing the at least one artificial intelligence model comprises:
parsing through the patient database to locate data associated with the patient; and
inputting the data associated with the patient into a patient report workflow to generate the patient report.

2. The system of claim 1, wherein the housing is transparent, wherein the interactive vial further comprises a display configured to display any one of a dosage schedule, a temperature of the interactive vial, and one or more alerts.

3. The system of claim 1, wherein the artificial intelligence model further comprises:
instructions to cross reference the data associated with the patient from the patient database with a global medical database to suggest improvements in a patient treatment plan;
instructions to predict when the patient needs to be notified about taking the prescribed medication; and
instructions to predict when the patient report needs to be communicated to a third-party entity.

4. The system of claim 1, wherein the offsite server is configured to transmit the generated patient report to any one of a doctor, a pharmacy, an emergency room, and a federal agency.

5. The system of claim 1, wherein the interactive vial further comprises:
a vial processor operable to transmit the data associated with the patient, wherein the vial processor utilizes quantum encryption to protect the patient data.

6. The system of claim 1, wherein the interactive vial further comprises:
at least one quantum temperature sensor configured to measure a temperature of the interactive vial, wherein the interactive vial is configured to issue an alarm if the temperature of the interactive vial exceeds a threshold.

7. The system of claim 1, wherein the interactive vial further comprises an internal alarm notification system configured to notify the patient of a missed dosage.

8. An interactive vial for controlling access to a medication, the interactive vial comprising:
a housing configured to receive and store the medication prescribed for a patient;
a controllable lock configured to inhibit access into the housing for a period of time; and
a vial processor configured to:
transmit instructions to the controllable lock to control access to the prescribed medication contained within the housing;
receive and compare a front side and a back side picture to a reference image for a medication shape, color, and logo, before setting a verification value to “YES” indicating that the medication matches the reference image for the prescribed medication; and
transmit patient data to an offsite server, wherein the offsite server is communicatively coupled to the interactive vial and is configured:
receive and store the patient data in a patient database;
use the patient data from the patient database as an input for an artificial intelligence model;
generate a patient report based on results generated from executing the artificial intelligence model; and
transmit the patient report to a third party entity.

9. The interactive vial of claim 8, wherein the housing is transparent, wherein the interactive vial further comprises a display configured to display any one of a dosage schedule, a temperature of the interactive vial, and one or more alerts.

10. The interactive vial of claim 8, wherein the artificial intelligence model is further configured to:
cross reference the patient data from the patient database with a global medical database to suggest improvements in a patient treatment plan;
predict when the patient needs to be notified about taking the prescribed medication; and
predict when the patient report needs to be communicated to the third party entity.

11. The interactive vial of claim 8, wherein the third party entity is any one of a doctor, a pharmacy, an emergency room, and a federal agency.

12. The interactive vial of claim 8, wherein the vial processor is configured to protect the patient data through quantum encryption.

13. The interactive vial of claim 8, wherein the interactive vial further comprises at least one quantum temperature sensor configured to measure a temperature of the interactive vial, wherein the interactive vial is configured to issue an alarm if the temperature of the interactive vial exceeds a threshold.

14. The interactive vial of claim 8, wherein the interactive vial further comprises an internal alarm notification system configured to notify the patient of a missed dosage.

15. A method for reporting results from an artificial intelligence model communicatively coupled to an interactive vial, comprising:
transmitting patient data from an interactive vial to an external server through a communication network wherein the external server comprises a memory storing at least one artificial intelligence model and a processor, communicatively coupled to the memory, configured to receive and store the patient data in a patient database;
executing the artificial intelligence model, wherein executing the at least one artificial intelligence model comprises:
parsing through the patient database to locate data associated with a patient; and
inputting the data associated with the patient into a patient report workflow to generate a patient report; and
transmitting the patient report to at least one third party entity.

16. The method of claim 15, wherein the artificial intelligence model further comprises:
instructions to cross reference the patient data from the patient database with a global medical database to suggest improvements in a patient treatment plan;
instructions to predict when the patient needs to be notified about taking a prescribed medication; and
instructions to predict when the patient report needs to be communicated to the third party entity.

17. The method of claim 15, further comprising measuring a temperature of the interactive vial with a quantum temperature sensor.

18. The method of claim 15, further comprising displaying one or more alerts on the interactive vial.
